(19) United States
(12) Patent Application Publication
(10) Pub. No.: US 2020/0368379 A1
(43) Pub. Date: Nov. 26, 2020

(54) MEDICAL PROBE DISINFECTANT SYSTEM

(71) Applicant: CIVCO Medical Instruments Co., Inc., Kalona, IA (US)

(72) Inventor: Yash Agarwal, New Haven, CT (US)

(21) Appl. No.: 16/880,555

(22) Filed: May 21, 2020

Related U.S. Application Data
(60) Provisional application No. 62/851,233, filed on May 22, 2019.

(51) Int. Cl. A61L 2/10; A61L 2/24; A61L 2/22; G16H 40/67; G16H 40/40; G06Q 30/06

(52) U.S. Cl. CPC A61L 2/10; A61L 2/24; A61L 2/22; G16H 40/67; G16H 40/40; A61L 2202/24; A61L 2202/11; A61L 2202/122; A61L 2202/15; A61L 2202/13; A61L 2202/14; G06Q 30/0633

(57) ABSTRACT

A medical probe disinfecting and sterilizing system for disinfecting and/or sterilizing medical devices may include a chamber for cleaning the probe by exposure to ultraviolet light and to a misted disinfectant chemical. The disinfectant may be misted by one or more ultrasonic transducers.

### CROSS REFERENCE TO RELATED APPLICATIONS

This Application claims priority under 35 U.S.C. § 119 to U.S. Provisional Patent Application No. 62/851,233 filed on May 22, 2019, entitled Medical Probe Disinfectant System, the contents of which are hereby incorporated herein by reference in their entirety

### BACKGROUND

Reusable medical devices that come in contact with a patient, such as ultrasound probes, must be disinfected between uses. Over the last several decades, the standard of cleaning and disinfecting heat sensitive reusable medical devices is a manual process. This generally involves soaking the medical device in a chemical disinfectant for a fixed time period at or above a minimum temperature. This is followed by rinsing, drying and storing the device before the next use.

Disadvantages of a manual process include time consumption; the need for operator training; risk of incomplete disinfection due to operator error; manual handling and exposure to strong chemicals such as glutaraldehyde, ortho-phthalaldehyde, or chlorine compounds; need for ventilation to reduce vapor concentration; need for manual neutralization and disposal of chemicals according to local and state regulations; degradation of the medical device with repeated and prolonged exposure to chemicals; and the need for manual record keeping. Exposure to chemical vapors is reduced by using a manual process Glutaraldehyde User Station (GUS), but the other disadvantages of the manual process remain.

Automated systems exist that allow a user to load a device, such as an ultrasound probe, into a system, press a button and disinfect the probe. Such automated systems typically use one of two different types of technologies for disinfection: chemical (hydrogen peroxide or peracetic acid/hydrogen peroxide blend) or ultraviolet light (UV) exposure.

Chemical based automated systems have disadvantages including: the use of high temperatures (sometimes in excess of 100° C./212° F.) to deliver chemicals in vapor form; longer cycle time (7+ mins); startup time; cost per cycle; and operator exposure to high temperature; probe damage due to heat and high concentration of disinfectant chemicals, such as 35% hydrogen peroxide solutions.

Existing UV based system offer advantages over the chemical based systems in that they do not use chemicals and have lower cycle time and costs. However, disadvantages of existing UV systems include: intensity of the UV light; high temperatures of up to 55° C./131° F.; high cost of UV bulbs and a need for periodic replacement; probe damage/wear with high intensity UV light; and difficulties in achieving even illumination across the device (e.g., shadowing).

### BRIEF DESCRIPTION OF THE DRAWINGS

FIG. 1 is a front view of an exemplary medical probe disinfectant system;

FIG. 2 is another front view of the exemplary medical probe disinfectant system of FIG. 1;

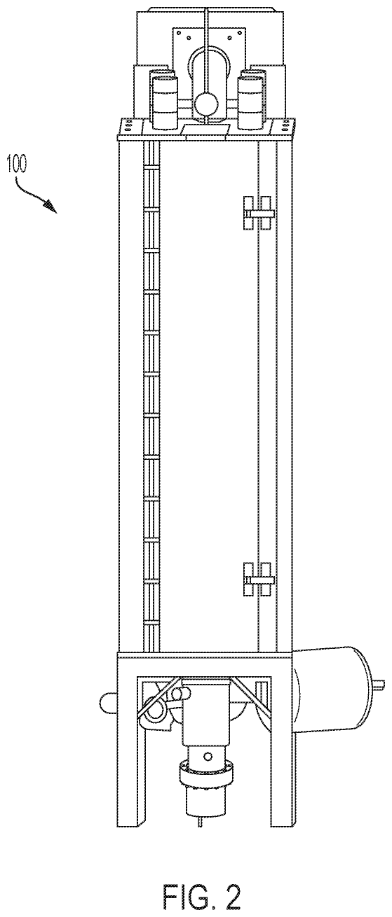

FIG. 3 is a side view of the exemplary medical probe disinfectant system of FIG. 1;

FIG. 4 is a rear view of the exemplary medical probe disinfectant system of FIG. 1;

FIG. 5 is a block diagram of an exemplary system controller;

FIG. 6 is a front view of another exemplary medical probe disinfectant system;

FIG. 7 is a right side view of the exemplary medical probe disinfectant system of FIG. 6;

FIG. 8 is a back side view of the exemplary medical probe disinfectant system of FIG. 6;

FIG. 9 is a top view of the exemplary medical probe disinfectant system of FIG. 6;

FIG. 10 is a front and left side view of the exemplary medical probe disinfectant system of FIG. 6;

FIG. 11 is a bottom view of the exemplary medical probe disinfectant system of FIG. 6;

FIG. 12 is a right side view of the exemplary medical probe disinfectant system of FIG. 6;

FIG. 13 is a rear view of the exemplary medical probe disinfectant system of FIG. 6;

FIG. 14 is a rear view of a portion of the exemplary medical probe disinfectant system of FIG. 6; and

FIG. 15 is a front view of a portion of the exemplary medical probe disinfectant system of FIG. 6.

### DETAILED DESCRIPTION OF PREFERRED EMBODIMENTS

Those skilled in the art will recognize other detailed systems and methods can be developed employing the teachings of the present invention. The examples provided here are illustrative and do not limit the scope of the invention, which is defined by the attached claims. The following detailed description refers to the accompanying drawings. The same reference numbers in different drawings may identify the same or similar elements.

In an exemplary embodiment of the invention, an automated system uses a combination of both UV light and chemicals to provide high level disinfection and sterilization of heat sensitive reusable medical devices.

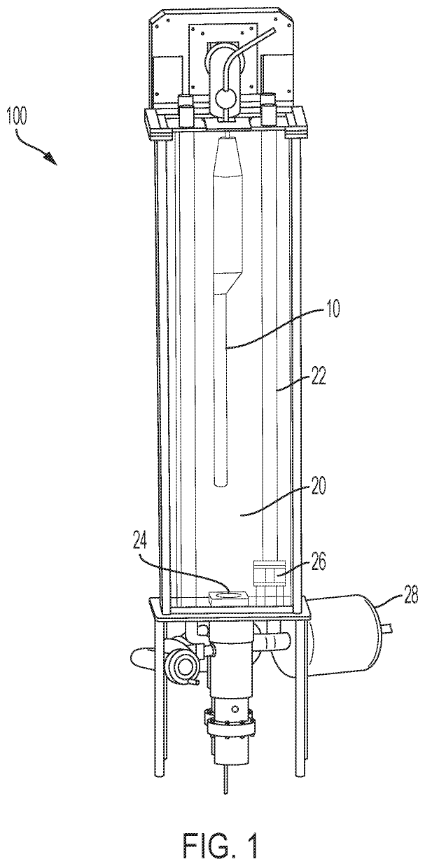

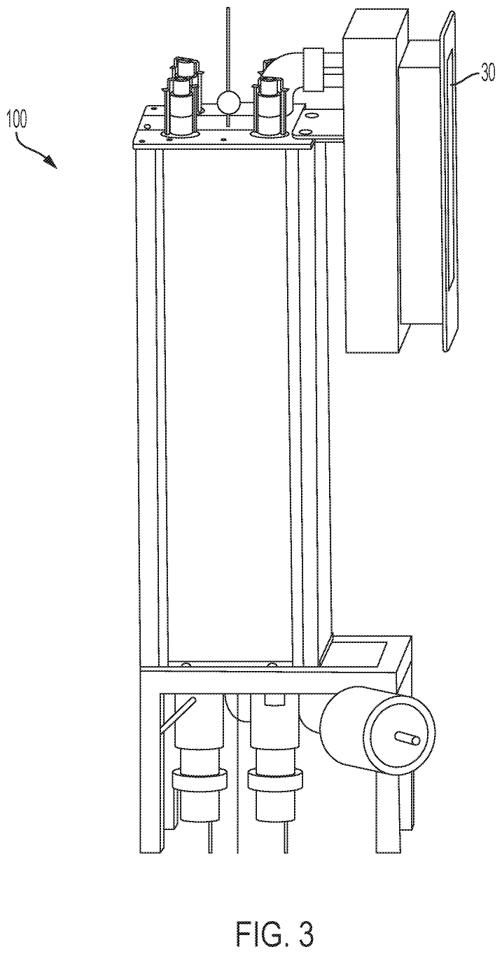

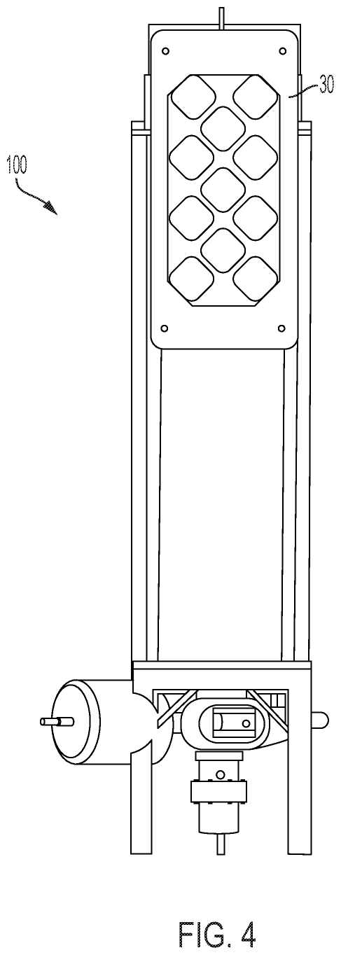

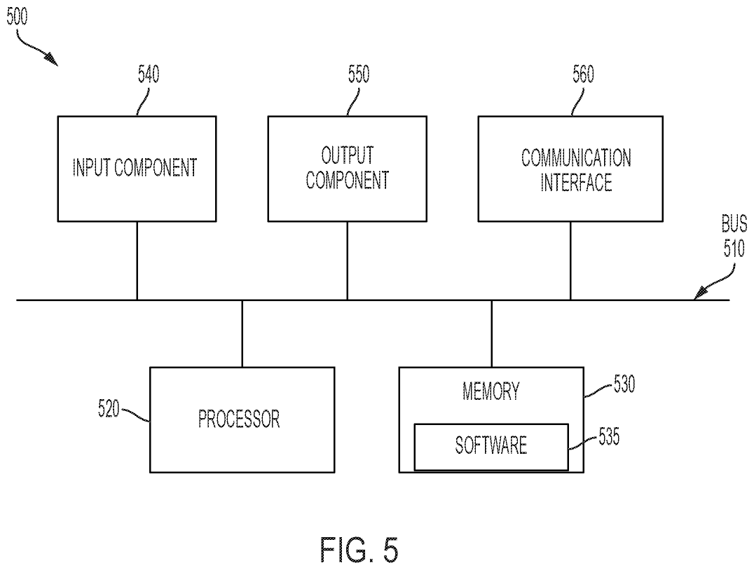

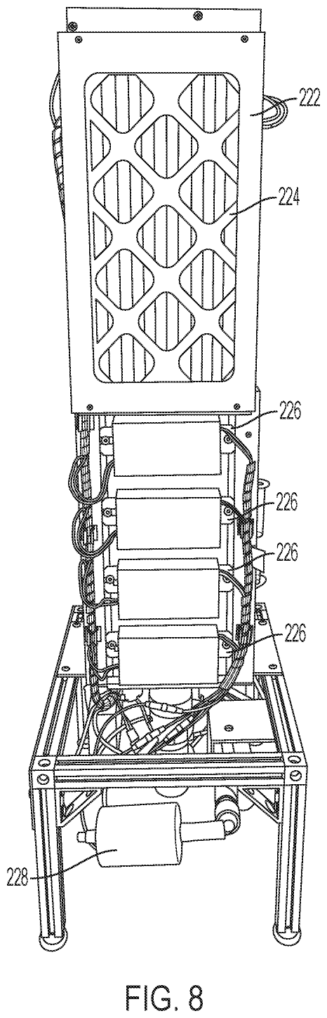

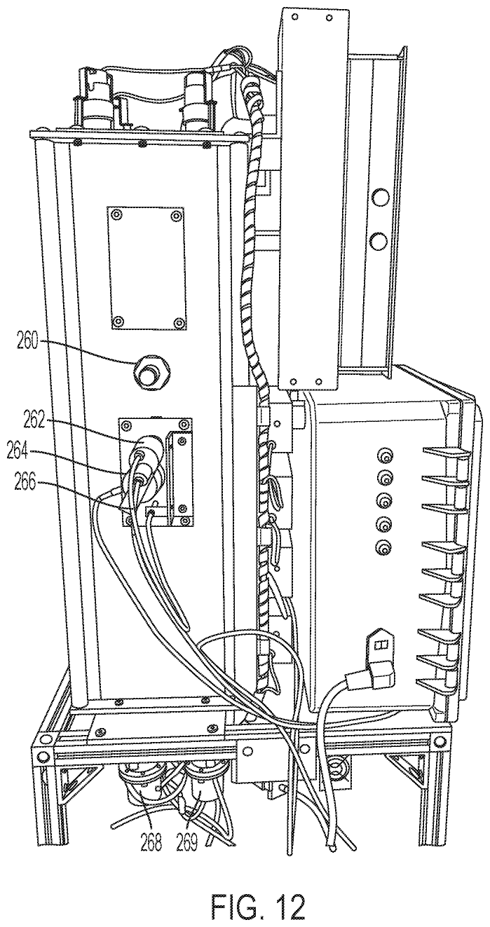

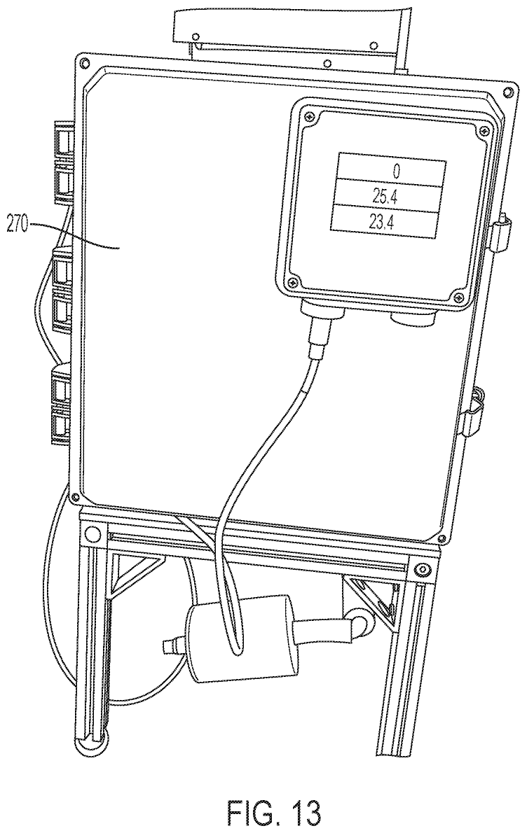

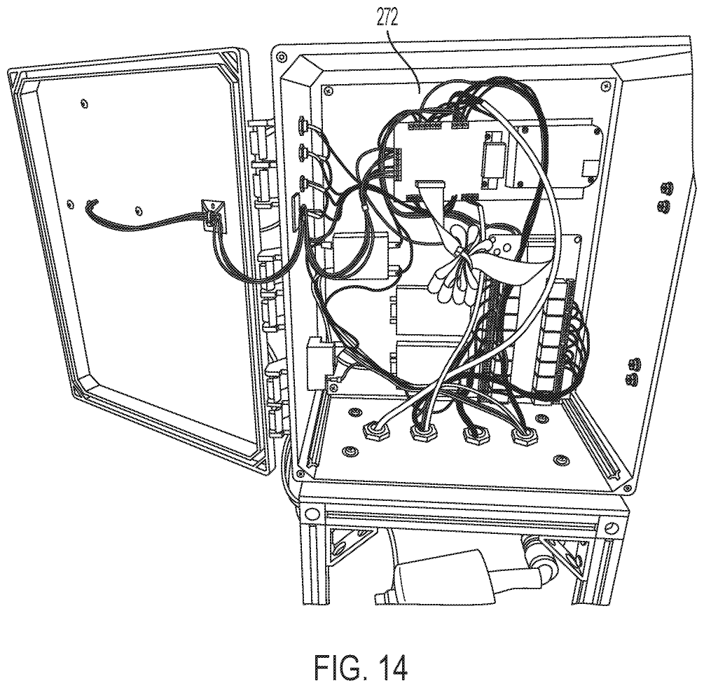

FIG. 1 shows a medical probe 10 within a chamber 20 of an exemplary disinfection system 100 according to an aspect of the invention. The probe 10 is suspended freely within the chamber and no part of the probe surface is in contact with any parts of the chamber 20. The probe 10 may be suspended, for example, in the chamber 20 by a cable or other mechanism that is attached to the probe 10, but not in contact with a patient. In an exemplary embodiment, at least four UV light sources 22 are arranged in an optimal fashion to provide 360° illumination of the probe 10 and provide a required minimum dose of UV light. The light sources may emit UV-B, UV-C or a combination of both. Consistent with embodiments described herein, the UV light sources 22 are protected from direct exposure to disinfectant chemical by a UV transparent barrier, such as a quartz tube. One or more ultrasonic transducers 24 are positioned within the chamber 20 to deliver, for example, a micron sized particle mist to the entire probe 10 surface and provide a required minimum dose of disinfectant. One or more fans 26 are included in the chamber 20 to evenly distribute the disinfectant mist. One or more pumps (see FIG. 12 description) deliver one or more chemicals to the one or more ultrasonic transducers 24. The system also includes one or more HEPA filters 228 shown in FIG. 8 to provide filtered air to carry the micron size particles from the ultrasonic transducers 24 to the chamber 20. The system 100 also includes a ventilation system including a filter 30 (shown in FIGS. 3 and 4) that filters any residual chemicals left in the chamber, neutralizes any residual disinfectant from exhaust air from said disinfecting chamber and maintains chemical vapor levels outside of the chamber within, for example, OSHA safety limits. The system 100 also includes sensors (see FIG. 12 and description below) to measure UV intensity, chemical concentration, ozone concentration, temperature, relative humidity and pressure. The system 100 also includes one or more controllers (see FIGS. 13 and 14 and their description below) to control the various components automatically and allow varying sequence and timing of operation of each component via an interface. FIG. 5 is a system block diagram of an exemplary controller 500, which is described in detail below. Chamber 20 and internal components exposed to UV and disinfectant are made of UV and chemically compatible materials.

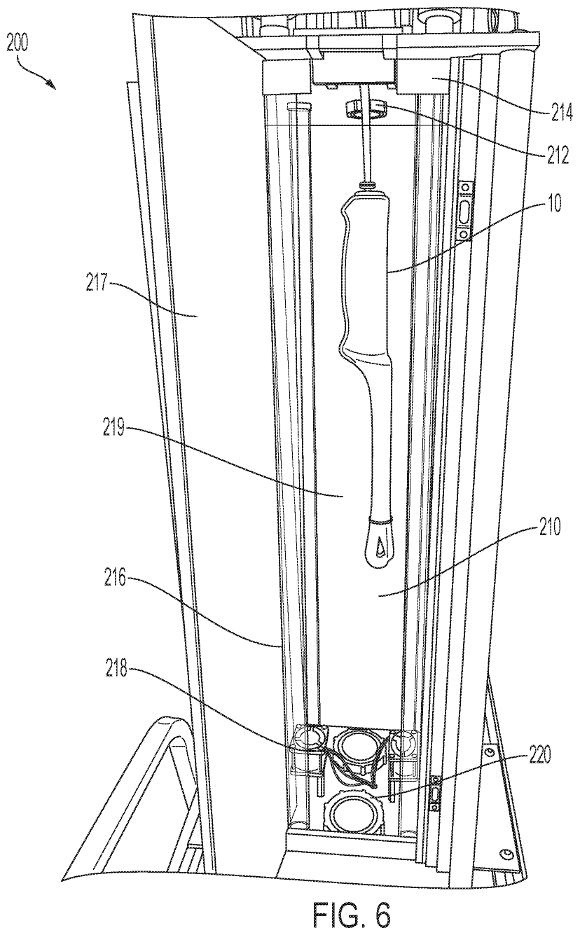

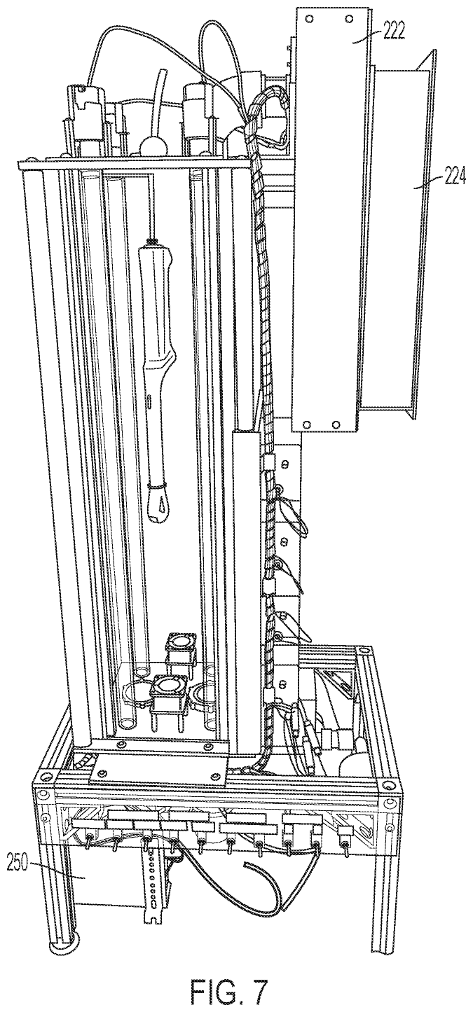

FIGS. 6-15 are various views of another embodiment of an exemplary medical probe disinfectant system 200. FIG. 6 is a front view the system wherein the following components are labeled: disinfecting chamber 210; exhaust port 212, upper circulation fan 214; quartz UV bulb (within a quartz tube) 216, lower circulation fan 218 and one or more ultrasonic misting transducers 220. FIG. 6 also shows a probe 10 loaded in chamber 210 and reflective walls labeled 217, 219. Note that all four walls of the chamber are reflective, only two walls are visible in FIG. 6. FIG. 7 is a right side view of the system (with a temporary transparent panel) of FIG. 6 wherein the following components are labeled: air diffuser box 222, activated carbon filter 224 and system power supply 250. FIG. 8 is a rear view of the system of FIG. 6 wherein the following components are labeled: air diffuser box 222, activated carbon filter 224; UV bulb ballasts and power supplies 226 and HEPA filter 228

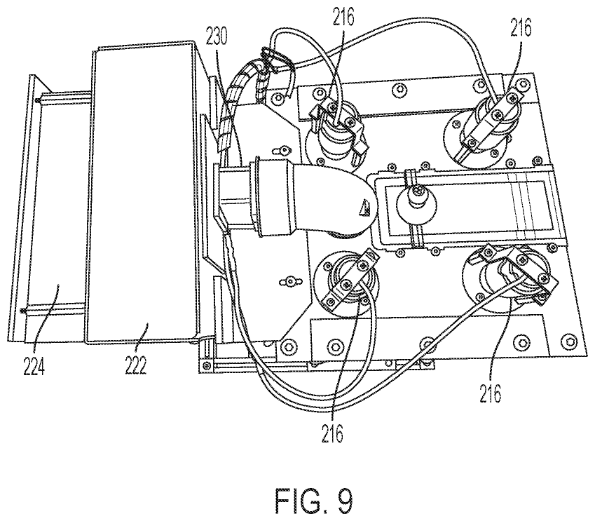

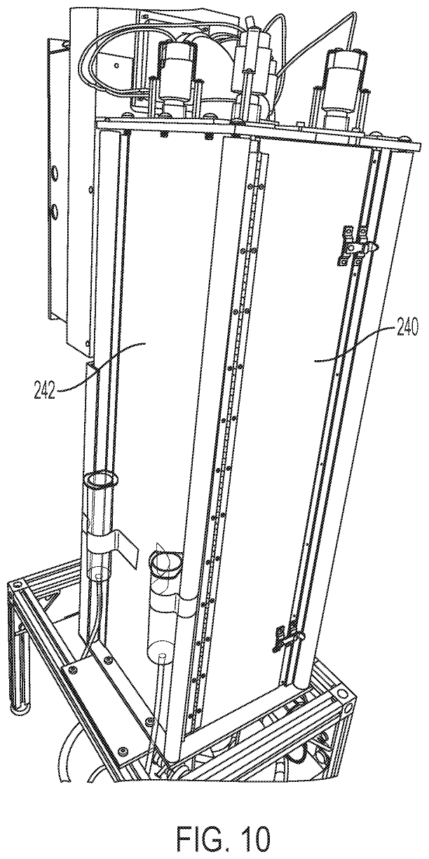

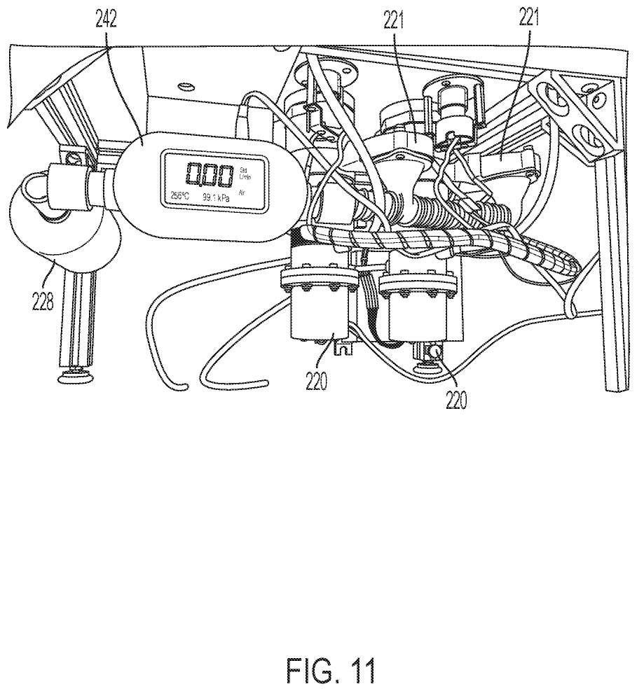

FIG. 9 is a top view of the system 200 of FIG. 6, with the following components labeled: tops of quartz UV bulbs 216; air diffuser box 222, activated carbon filter 224 and exhaust fan 230. FIG. 10 is a front and left side view of the system 200 of FIG. 6 showing the doors 240 and 242 in place to shield users from UV light. FIG. 11 is a bottom view of the system 200 of FIG. 6 with following components labeled: air pressure gauge 242, ultrasonic misting transducers 220, misting engine fans 221 and HEPA filter 228. FIG. 12 is a right view of the system 200 of FIG. 6, with the following components labeled: ozone sensor 260, hydrogen peroxide and relative humidity sensor 262, UV light sensor 264, temperature sensor 266 and disinfectant chemical pumps 268, 269. FIGS. 13 and 14 are rear views of the system 200 of FIG. 6 showing the controller enclosure 270 and controller interior 272, respectively.

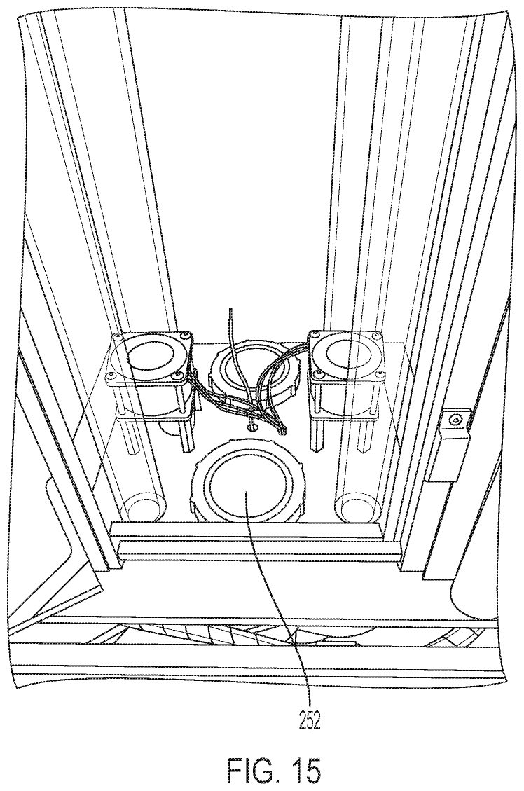

FIG. 15 shows a partial front view of the system 200 of FIG. 6 with a wire mesh 252 on top of a misting transducer.

In exemplary embodiments, system 100 and/or 200 may also include a pre-clean system to eliminate the need for pre-cleaning the reusable medical device by the user. The system may also accommodate multiple probes 10 and disinfect or sterilize the probes simultaneously. Multiple probe systems may include the ability to allow asynchronous operation, i.e., add remove a probe without disturbing a disinfection cycle of another probe, by the use of partitioned disinfecting chambers. The system may also accommodate a probe cable and connector and disinfect/sterilize the probe cable and connector, as well as the probe. The system may have wired or wireless network connectivity and allow for integration into the operating facility's electronic healthcare information system (HIS) or electronic medical records (EMR) systems. The system may automatically neutralize any chemicals after use and not require the user to handle chemicals and/or have zero residue, zero emissions and zero waste. The system may include chemical sensors that provide the ability to automatically verify the concentration of a disinfectant solution before use.

In further embodiments, the system may automatically verify concentration or dosage of disinfection technology. The system may automatically verify that the cycle parameters are within specification and provide a verification of efficacy at the end of each cycle either by digital reading of various sensors, a chemical indicator, a biological indicator or by a combination thereof. The system may provide cleaning efficacy before and after cycle, for example using adenosine triphosphate. The system may have cameras or other sensors combined with smart algorithms or artificial intelligence to inspect the probes for any defects before disinfection. The system may store probe inspection history along with images to provide a history of probe degradation over lifetime. The system may perform automated electrical leak testing. The system may allow logging of use and ordering of consumables. The system may perform automatic drying if the disinfectant is such that this is required. The system may automatically add a sterile cover on the probe after disinfection and drying or a coating such as Zwitterion particles. The system may allow probe storage after reprocessing. The system may automatically control any parameters (time, temperature, concentration, removal of residual high level disinfectant). The system may provide digital traceability of cleaned probes. The system may provide a software interface to download specific disinfecting cycles based on date range. The system may be configured to allow a user to interrupt the disinfection cycle and safely remove the probe without being exposed to chemicals or UV light. The system may provide a printed record of the disinfecting cycle.

In some embodiments, a system as described herein may include a self-test/self-calibration option to check before first use and periodic check/calibration of sensors. The system may include a display and provide training video(s) on the system display both at initial startup as well as on-demand training. Systems with this capability may also provide training competency testing and store training logs. The system may be configured to integrate a probe transport case or have the disinfection chamber be the transport case. The system may have the capability to link multiple probe transport cases together.

The system may also be configured to clean general purpose (GP) (i.e., surface), transesophageal echocardiography (TEE) probes, other non-lumened scopes used in Urology and Gastroenterology, needle guides, brackets, scalpels, scopes, etc.

The system may have an automated diagnostic system and provide storage of service logs. The system may enable remote service and troubleshooting. The system may include a service toolkit within the unit for frequently used tools. The system may use similar types of hardware to minimize tool requirements. The system design will be portable and allow it to operate safely anywhere within a facility where an electrical connection is available.

User interfaces to the system may include: gesture recognition, voice activated operation, a touch screen, audio, visual indicator lights visible from across a room and remote status/control capability whereby users can see the status, run troubleshooting, diagnostics, download logs etc.

Chemical cleaning consistent with embodiments of the invention may user various chemical reactions for the cleaning. For example, in one embodiment, photons in the deep UV range will typically break the O—O bond in the middle of the hydrogen peroxide molecule, releasing two Hydroxyl radicals. The oxidation potential of the Hydroxyl radical is 2.80 Volts (higher as compared to Ozone 2.07, Hydrogen Peroxide 1.78V, Chlorine dioxide 1.58, Chlorine 1.36 and Oxygen molecule 1.26), thus increasing the likelihood of faster kill of bacteria.

The chemicals will absorb better as the wavelengths decrease into UV-C and absorb less as wavelengths approach the visible range. Optically, considerable scattering of the UV light in the disinfection cavity occurs when the aerosol mist is present. Such scattering may increase the even distribution of the UV light illumination incident on most surfaces regardless of orientation. Along with the scattering, incident light may exhibit longer wavelengths, as shorter wavelengths are preferentially absorbed. This may result in the liberation of Hydroxyl radicals that become available for surface disinfection.

Consistent with embodiments described herein, a system that automatically combines UV and chemical disinfection may, when compared to a manual process, automates the process and removes causes of operator error, variability, and improves operator and patient safety. Furthermore, as compared to the automated systems using a single technology, embodiments described herein kill microorganisms via multiple pathways in a single system, i.e., UV, Chemical and Ozone produced during UV generation, all of which are independently proven to be efficacious.

Multiple pathways reduce likelihood of repair mechanisms of the microorganism and thus increase probability of complete kill. In addition, the system described herein may provide faster kill of microorganism (possibly instantly) and may provide high level disinfection in a shorter time period as compared to chemical only disinfection (8 mins) and UV only disinfection (2-4 minutes). Consistent with embodiments described herein, a multimodal system may provide sterilization in a relatively shorter time period as compared to liquid chemical (2+ hours). Accordingly, expected probe damage may be lower due to lower dosage and exposure times,

The above-described system may provide the benefit of both UV and Chemical disinfection technologies without the disadvantages, such as: Lower intensity/dose UV light (thus longer bulb life and maybe adapted to using UV LEDs with theoretically no bulb changes required during the life of the product) and lower concentration/dose of active chemical ingredients. Furthermore, systems described herein may eliminate the need for a catalytic destruction of hydrogen peroxide and rinsing of the medical device, since UV light “evaporates” the active ingredient in chemical or breaks the chemicals down into harmless components such as oxygen or water.

The above described system may also operate at room temperature, which is a noted advantage over either chemical or UV independent solutions.

FIG. 5 is a diagram illustrating exemplary physical components of a device 500. Device 500 may correspond to various devices within the above-described systems 100 and 200, such as the system controller. Device 500 may include a bus 510, a processor 520, a memory 530, an input component 540, an output component 550, and a communication interface 560.

Bus 510 may include a path that permits communication among the components of device 500. Processor 520 may include a processor, a microprocessor, or processing logic that may interpret and execute instructions. Memory 530 may include any type of dynamic storage device that may store information and instructions, for execution by processor 520, and/or any type of non-volatile storage device that may store information for use by processor 520.

Software 535 includes an application or a program that provides a function and/or a process. Software 535 is also intended to include firmware, middleware, microcode, hardware description language (HDL), and/or other form of instruction. By way of example, with respect to the network elements that include logic to provide proof of work authentication, these network elements may be implemented to include software 535. Additionally, for example, device 500 may include software 535 to perform tasks as described above such as automated disinfecting operation, remote operation, self-diagnostics and remote diagnostics.

Processing in an exemplary disinfection system may include: connectivity for integration into the operating facility's HIS or EMR system; automated verification of concentration or dosage of disinfection technology; automated verification that the cycle parameters were within specification and provide a verification of efficacy at the end of each cycle; determination of cleaning efficacy before and after cycle; storage of probe inspection history along with images to provide a history of probe degradation over lifetime; automated electrical leak testing; logging of use and ordering of consumables; automated control of system parameters such as time, temperature, concentration and removal of residual high level disinfectant; implement digital traceability of cleaned probes; ability for user download of specific disinfecting cycles based on date range; and print capability for record of the disinfecting cycle.

In some embodiments, a system as described herein may include a self-test/self-calibration option to check before first use and periodic check/calibration of sensors. In such embodiments, device/system controller 500 may operate the self-test/self-calibration. The system may include a display and provide training video(s) on the system display both at initial startup as well as on-demand training. Systems with this capability may also provide training competency testing and store training logs. The system may be configured to integrate a probe transport case or have the disinfection chamber be the transport case. The system may have the capability to link multiple probe transport cases together.

Input component 540 may include a mechanism that permits a user to input information to device 500, such as a keyboard, a keypad, a button, a switch, a touch screen, voice commands, gestures, camera, miniature radar, time of flight sensor, etc. Output component 550 may include a mechanism that outputs information to the user, such as a display, a speaker, one or more light emitting diodes (LEDs), printer etc.

Communication interface 560 may include a transceiver that enables device 500 to communicate with other devices and/or systems via wireless communications, wired communications, or a combination of wireless and wired communications. For example, communication interface 560 may include mechanisms for communicating with another device or system via a network. Communication interface 560 may include an antenna assembly for transmission and/or reception of radio frequency (RF) signals. In one implementation, for example, communication interface 560 may communicate with a network and/or devices connected to a network. Alternatively or additionally, communication interface 560 may be a logical component that includes input and output ports, input and output systems, and/or other input and output components that facilitate the transmission of data to other devices.

Device 500 may perform certain operations in response to processor 520 executing software instructions (e.g., software 535) contained in a computer-readable medium, such as memory 530. A computer-readable medium may be defined as a non-transitory memory device. A non-transitory memory device may include memory space within a single physical memory device or spread across multiple physical memory devices. The software instructions may be read into memory 530 from another computer-readable medium or from another device. The software instructions contained in memory 530 may cause processor 520 to perform processes described herein. Alternatively, hardwired circuitry may be used in place of or in combination with software instructions to implement processes described herein. Thus, implementations described herein are not limited to any specific combination of hardware circuitry and software.

Device 500 may include fewer components, additional components, different components, and/or differently arranged components than those illustrated in FIG. 5. As an example, in some implementations, a display may not be included in device 500. In these situations, device 500 may be a “headless” device that does not include input component 540. Additionally, or alternatively, one or more components of device 500 may perform one or more tasks described as being performed by one or more other components of device 500.

In an exemplary implementation, one or more components described above may perform operations in response to its respective processor (or processors) (e.g., processor 520) executing sequences of instructions contained in a non-transitory computer-readable medium. A computer-readable medium may be defined as a physical or logical memory device. The software instructions may be read into memory from another computer-readable medium (e.g., a hard disk drive (HDD), solid state drive (SSD), etc.), or from another device via a communication interface. Alternatively, hard-wired circuitry may be used in place of or in combination with software instructions to implement processes consistent with the implementations described herein. Thus, implementations described herein are not limited to any specific combination of hardware circuitry, firmware and software.

Although the invention has been described in detail above, it is expressly understood that it will be apparent to persons skilled in the relevant art that the invention may be modified without departing from the spirit of the invention. Various changes of form, design, or arrangement may be made to the invention without departing from the spirit and scope of the invention. Therefore, the above-mentioned description is to be considered exemplary, rather than limiting, and the true scope of the invention is that defined in the following claims.

No element, act, or instruction used in the description of the present application should be construed as critical or essential to the invention unless explicitly described as such. Also, as used herein, the article “a” is intended to include one or more items. Further, the phrase “based on” is intended to mean “based, at least in part, on” unless explicitly stated otherwise.

What is claimed is:

1. A system for disinfecting or sterilizing a medical device comprising:
a disinfecting chamber configured to house a probe;
one or more ultraviolet light sources located inside said disinfecting chamber with reflective panels;
one or more ultrasonic misting transducers with fans to generate and circulate a disinfectant chemical mist; and
a pump for providing the chemical to said ultrasonic misting transducers.

2. The system of claim 1, further comprising:
at least two reflective panels within said disinfecting chamber.

3. The system of claim 1, further comprising:
a HEPA air filter configured to filter air provided to said disinfecting chamber.

4. The system of claim 1, further comprising:
an exhaust air filter configured to neutralize any residual disinfectant chemical from exhaust air from said disinfecting chamber.

5. The system of claim 4, wherein said exhaust air filter is an activated carbon filter.

6. The system of claim 1, further comprising:
at least one air circulation fan and one exhaust fan.

7. The system of claim 1, further comprising:
one or more sensors configured to sense at least one of: UV intensity, disinfectant chemical concentration, ozone concentration, temperature, relative humidity or pressure within said disinfecting chamber.

8. The system of claim 1 wherein said UV light source is a UV-B or a UV-C light source.

9. A method of disinfection or sterilization of a medical device comprising:
providing a source of ultraviolet light;
providing a disinfectant chemical;
creating a micron-sized mist of said disinfectant chemical; and
simultaneously or sequentially exposing, within a chamber, the medical device to said UV light and said micron-sized mist of disinfectant chemical.

10. The method of claim 9, wherein the medical device is a non-lumened medical device.

11. The method of claim 9, further comprising:
circulating with a fan said micron-sized mist within said chamber.

12. The method of claim 11, further comprising:
filtering intake air for said circulating with a HEPA filter.

13. The method of claim 9, further comprising:
monitoring levels of said UV light and said chemical disinfectant with one or more sensors.

14. The method of claim 9, further comprising:
tracking the disinfection or sterilization of the medical device on an operating facility's healthcare information system (HIS) or electronic medical records (EMR) system.

15. The method of claim 14, further comprising:
automatically verifying concentration of said disinfectant chemical or dosage of said UV light on said operating facility's HIS or EMR system.

16. The method of claim 9, further comprising:
verifying that disinfection or sterilization cycle parameters were within specification at the end of a disinfection or cleaning cycle.

17. The method of claim 16, wherein said verifying is performed on on an operating facility's HIS or EMR system.

18. The method of claim 9, further comprising:
storing medical device inspection history along with images to provide a history of medical device degradation.

19. The method of claim 18, wherein said storing is performed on on an operating facility's HIS or EMR system.

20. The method of claim 14, further comprising:
automated logging of use and ordering of consumables associated with disinfections and sterilizations of medical devices.
